(19) United States
(12) Patent Application Publication
(10) Pub. No.: US 2022/0105217 A1
(43) Pub. Date: Apr. 7, 2022

(54) ROOM SANITIZER

(71) Applicant: Bruce Hinkle, Newport Beach, CA (US)

(72) Inventor: Bruce Hinkle, Newport Beach, CA (US)

(21) Appl. No.: 17/489,412

(22) Filed: Sep. 29, 2021

Related U.S. Application Data
(60) Provisional application No. 63/086,416, filed on Oct. 1, 2020.

(51) Int. Cl. A61L 2/20; C01B 13/11

(52) U.S. Cl. CPC A61L 2/202; C01B 13/11; C01B 2201/90; A61L 2202/14; A61L 2202/11

(57) ABSTRACT

A room sanitizer has an ozone generator unit having an ozone generator fan. A control unit controls the ozone generator unit. An occupancy sensor connected to the control unit shuts down the ozone generator when the occupancy sensor is activated. The control unit controls an ozone exhaust fan. An ozone destruct module holds ozone destruct catalyst and is connected to the ozone exhaust fan. A remote control is connected to a remote control connector that is electrically connected to the control unit. The remote control further includes a power switch having a power switch warning light, an ozone generator light, and a safe entry light. A remote control cable harness connects the remote control to the control unit. The control unit has an ozone timer relay setting a time for operating the ozone generator, and an ozone destruct timer relay setting a time for operating the ozone exhaust fan.

This application claims priority from U.S. provisional application 63/086,416 for room sanitizer, by inventor Bruce Hinkle filed Oct. 1, 2020, the disclosure of which is incorporated herein by reference.

### FIELD OF THE INVENTION

The present invention is in the field of room sanitizers.

### BRIEF DISCUSSION OF RELATED ART

Because of coronavirus pandemics, room sanitizing has become popular and extends to hospital rooms, offices, homes and in particular hotel rooms that require sanitizing between guests.

### SUMMARY OF THE INVENTION

A room sanitizer has an ozone generator unit having an ozone generator fan. A control unit controls the ozone generator unit. An occupancy sensor connected to the control unit shuts down the ozone generator when the occupancy sensor is activated. The control unit controls an ozone exhaust fan. An ozone destruct module holds ozone destruct catalyst and is connected to the ozone exhaust fan. A remote control is connected to a remote control connector that is electrically connected to the control unit. The remote control further includes a power switch having a power switch warning light, an ozone generator light, and a safe entry light. A remote control cable harness connects the remote control to the control unit. The control unit has an ozone timer relay setting a time for operating the ozone generator, and an ozone destruct timer relay setting a time for operating the ozone exhaust fan.

### BRIEF DISCUSSION OF THE DRAWINGS

FIG. 1 is a diagram of a room sanitizer.

FIG. 2 is an electrical diagram of the room sanitizer.

The following call out list of elements can be a useful guide in referencing the element numbers of the drawings.
20 Remote Control
21 DIN Connector
22 Front
23 Power Switch Warning Light
24 Ozone Generator Light
25 Safe Entry Light
26 Remote Control Housing
27 Switch Connector
28 Ozone Light Connector
29 Safe Entry Light
30 12 VDC Ozone Timer Relay
31 12V Ozone GN Blue Light Relay
32 O3 Ozone Generator Element
33 First 12 VDC/+Block
34 Second 12 VDC/−Block
35 Third 12 VDC/+Block
36 12 VDC/+Transformer
37 DPDT Relay to 12 VDC Transformer
38 Occupancy Sensor
39 12 VOC Alarm
40 12 VDC Ozone Destruct Time Relay
41 Ozone Exhaust Fan
42 Relay 110 VAC Alarm on/off
50 12 VDC Safe Entry Timer Relay
60 Main Switch
61 Ground Fault Interrupt
62 Plug Inlet
63 Main Switch Light
64 Ground
65 Ozone Generator Ground
66 Ozone Exhaust Fan Ground
67 GFCI Ground Line
68 Plug Ground
69 Neutral Block Supply
70 Neutral AC Block
71 Occupancy Sensor Neutral
72 Alarm Relay Neutral
73 Transformer Relay Neutral
74 Transformer Neutral
75 Destruct Timer Relay Neutral
76 Ozone Light Relay Neutral
77 Ozone Neutral
78 Exhaust Fan Neutral
79 Neutral In
80 Load Block
81 Plug Load Line
82 GFCI Load Line
83 Switch Load Line
84 Sensor Load
85 Load Block Load Input Line
86 First Relay Load Line
87 Second Relay Load Line
88 Ozone Timer Destruct Relay Load Line
89 Ozone Light Relay Load Line
90 Room Unit
91 Ozone Generator Air intake
92 Ozone Destruct Air Intake
93 Destruct Ducting
94 Ozone Destruct Module
95 Ozone Generator Air Filter
96 Remote Connector
97 Ozone Generator Fan
98 Ozone Generator Unit
99 Control Box
100 Catalyst
101 Catalyst Air Filter
102 Catalyst Air Intake Opening
103 Air Intake Grille
104 Upper-Level
105 Lower-Level
106 Lower-Level Side Air Filter
107 Lower-Level Side Air Filter Slot
108 Cable Harness

### DETAILED DESCRIPTION OF THE PREFERRED EMBODIMENTS

Glossary

GN is short for generator

GFI is a common abbreviation for ground fault interrupt

VAC is an abbreviation for alternating current voltage

VDC is an abbreviation for direct current voltage

DIN is an abbreviation for Deutsches Institut für Normung, which translates to German Institute for Standardization and is a type of electrical connector

DPDT is an abbreviation for dual pole dual throw which is a type of electrical relay

HEPA stands for high-efficiency particulate air

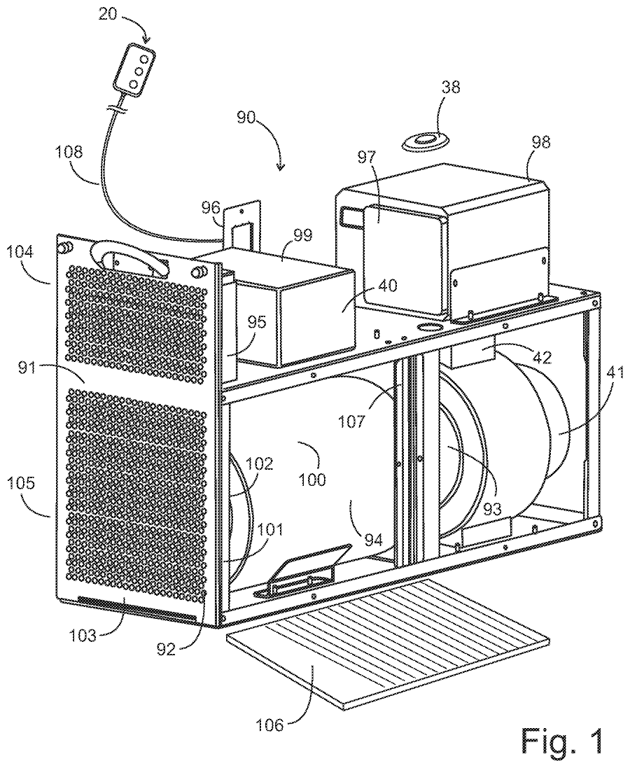

As seen in FIG. 1, a room unit 90 has an ozone generator 98 that operates in conjunction with an ozone generator fan 97 in a first mode. The first mode is the ozone generation mode where ozone is generated from oxygen. During the ozone generation, the ozone generator air intake 91 intakes air and an air filter such as a HEPA air filter provides filtration of air entering the ozone generator fan 97 and passing through the ozone generator unit 98. The HEPA air filter can be pleated. The control unit is preferably formed as a control box 99. The control box 99 and remote control 96 are in line to the ozone generation airflow. Intake air flow passes through the ozone generator air intake 91 first, then the ozone generator air filter 95, then the control box 99, then the remote connector 96, then the ozone generator fan 97, and then the ozone generator unit 98. In this way, the flow of air passes over the control box 99 for air convection cooling. Also, the 12 VDC ozone destruct time relay 40 can be mounted in the control box 99 for air convection cooling.

In a second mode, the room sanitizer catalyzes the ozone back to oxygen. The room unit 90 has an ozone exhaust fan 41 is mounted in line with the ozone destruct module 94 via destruct ducting 93. A relay 110 VAC alarm on/off 42 can be mounted near the ozone exhaust fan 41. The destruct ducting 93 and the ozone destruct module 94 can be made of stainless steel. The ozone exhaust fan 41 is preferably an in-line fan. The ozone generator air intake 91 and the ozone destruct air intake 92 can share a common grill formed from a perforated metal plate. The perforated metal plate can be removed for cleaning. The ozone destruct air intake 92 is in line to the catalyst air filter 101, ozone destruct module 94, the catalyst 100 of the ozone destruct module 94, then the destruct ducting 93, then the ozone exhaust fan 41. The catalyst air filter 101 is placed in front of a catalyst air intake opening 102. The catalyst air filter 101 can be a rectangular air filter that fits in a slot retained by the air intake grille 103.

The air filter 95 for the ozone generator can optionally be installed in line to the ozone destruct air intake 92. Preferably, the remote connector 96 is electrically connected to the control box 99, and the remote control can connect to the remote connector 96. The catalyst 100 can be a zeolite honeycomb, manganese dioxide pellets, or the like. The filter media is packed into a face of the ozone destruct module 94. The ozone generation apparatus in the first mode can be on the upper level 104, and the ozone destruct apparatus in the second mode can be on the lower level 105. The lower level preferably has a lower-level side air filter 106. An outer cover can fit over the upper level 104, and the lower level 105. A lower-level side air filter 106 can fit into a lower-level side air filter slot 107. The cable harness 108 can be a flat cable such as a ribbon cable.

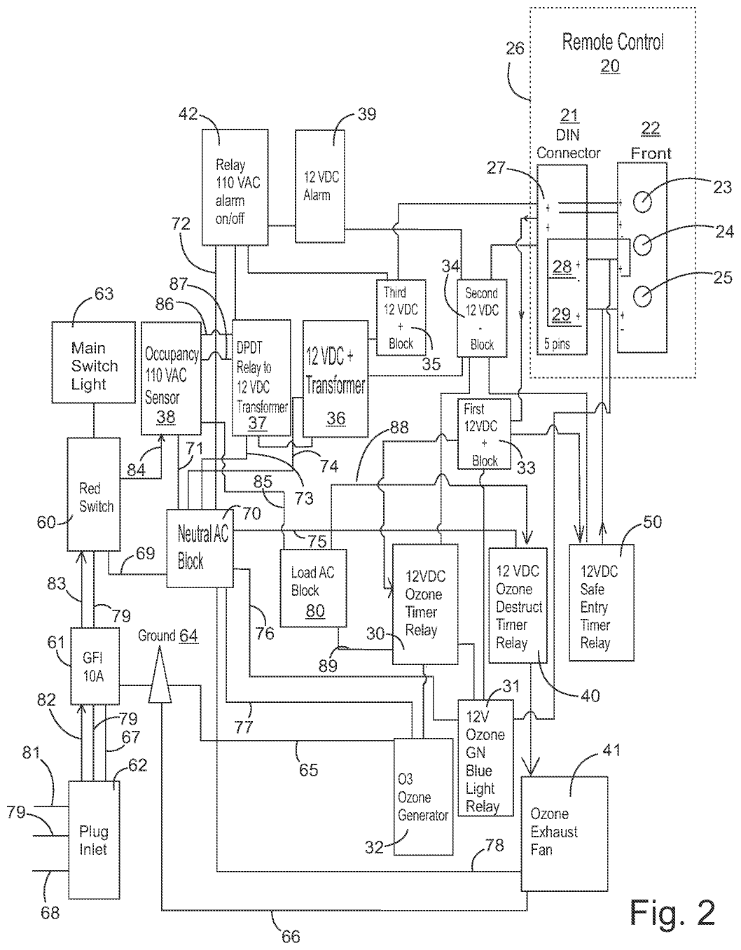

As seen in FIG. 2, a remote control 20 can be connected by a din connector 21 to the remote connector 96 near the control box 99. A front 22 of the remote control housing 26, of the remote control 20 preferably includes a power switch warning light 23, ozone generator light 24, and a safe entry light 25. A switch connector 27 preferably connects the power switch warning light to the control box 99. The power switch warning light has a combined switch and light. When a user presses the power switch, the power switch illuminates preferably with a red light. The remote control 20 is not wireless, but has a cable harness 108 that is configured to pass under a door so that the user can control the unit from outside of the room. The cable harness is preferably sheathed in a bundle.

The ozone light connector 28 on the din connector 21 connects the remote connector 96 to the ozone generator light 24. When the user activates the ozone generator in the first mode, the ozone generator light 24 is activated. Preferably, the ozone generator light 24 is a blue light. The ozone generator has an ozone timer relay that can be formed as 12 VDC ozone timer relay 30. The ozone timer relay provides an activation duration of the ozone generator.

The safe entry light connector 29 on the din connector 21 connects the remote connector 96 to the safe entry light 25. When the second mode is complete, the safe entry light 25 activates to indicate that the room is safe to reenter. Preferably, the safe entry light 20 is a green light. A safe entry light relay activates the safe entry light 20 and the safe entry light relay can be formed as the 12 v ozone GN blue light relay 31.

The ozone destruct time relay formed as the 12 VDC ozone destruct time relay 40 sets a time for the operation of the second mode.

The occupancy sensor can be infrared, ultrasonic, and operate on 110 VAC. While a user is in the room, the occupancy sensor does not allow the power switch on the remote control 22 activate the ozone generator. The occupancy sensor cuts off power to the AC load block whenever it detects occupancy to shut down the ozone generator unit 98 and all other components such as the ozone exhaust fan 41, and the remote control 20. The ozone generator unit 98 has an ozone generator element 32 which can be formed as corona discharge, electrostatic plates or the like. The ozone generator element 32 is deactivated when the occupancy sensor 38 toggles the DPDT relay to 12 vdc transformer 37. The activation of the relay alarm, formed as relay 110 vac alarm on/off 42 activates the alarm formed as the 12 voc alarm 39. The alarm is preferably an audio and visual indication such as a siren and flashing light.

The room unit 90 has an electrical organization based on a ground block 64, a neutral AC block 70, and a load block. The plug inlet 62 connects to mains power by plug load line 81, neutral line 79, and plug ground 68. The plug inlet 62 connects to the ground fault interrupt 61 which can be a ten amp circuit such as a 10 A GFCI via a GFCI load line 82, neutral line 79, and a GFCI ground line 67.

A ground block formed as ground 64 provides an ozone generator ground 65 to the ozone generator, and provides an ozone exhaust fan ground 66 to the ozone exhaust fan. The ground fault interrupt 61 is connected to the main switch 60 by a switch load line 83, and a neutral line 79. The main switch 60 may include or can be connected to the main switch light 63. The main switch 60 connects to the occupancy sensor 38 at the sensor load line 84.

The load block 80 preferably has a load block load input line 85 connected to the occupancy sensor 38. The load block 80 then provides load lines to a variety of different components including the ozone timer destruct relay load line 88 which connects to the ozone destruct timer relay 40, and the ozone light relay load line 89 which connects to the ozone timer relay 30.

A first relay load line 86 and a second relay load line 87 connects from the occupancy sensor to the DPDT Relay to 12 VDC Transformer 37.

A first 12 vdc/+block 33, second 12 vdc/−block 34, and third 12 vdc/+block 35 provide toehold power from the 12 vdc/+transformer 36 to a the remote control 20, the alarm 39, and the safe entry timer relay 50 formed as a 12 vdc safe entry timer relay 50.

A neutral block provides a neutral line for relevant components and can be formed as a neutral AC block 70. The neutral block supply 69 provides a neutral connection to the neutral AC block 70. The occupancy sensor neutral 71 provides a neutral line to the occupancy sensor 38. The alarm relay neutral 72 provides a neutral line to the alarm relay 42. A transformer relay neutral 73 provides a neutral line to the DPDT relay to 12 VDC transformer 37. A transformer neutral 74 provides a neutral line to a 12 VDC/+Transformer. A destruct timer relay neutral 75 provides a neutral line to the 12 VDC ozone destruct timer relay 40. An ozone light relay neutral 76 provides a neutral line to the ozone generator light relay formed as the 12V Ozone GN Blue Light Relay 31. An ozone neutral 77 provides a neutral line to an ozone generator 32. An exhaust fan neutral 78 provides a neutral line for the ozone exhaust fan 41.

Operation of the room sanitizer begins with moving the room sanitizer to a hotel room. The room sanitizer is preferably mounted on caster wheels with a pull handle mounted on the housing. A user can pull the room sanitizer into a hotel room and plug it in. After plugging in the room sanitizer, the occupancy sensor will activate and detect the user inside the room. The activation of the occupancy sensor disables the operation of the room sanitizer. The user pulls the cable harness under the door so that the remote control protrudes from underneath the hotel door. The user can then press the power on button on the sanitizer remote control. The power on button will light with a red light. The ozone active light such as the blue light will activate for 30 minutes the first mode of operation. The second mode of operation, the ozone destruct will operate for 30 minutes. During this time, the ozone active light will remain lit. After the second mode of operation, the safe entry light will activate to show that the room is safe for reentry. With this construction, the user only has a single button to press to activate the room sanitizer, and the room sanitizer operates automatically during sanitation processes.

1. A room sanitizer comprising:
a. an ozone generator unit, wherein the ozone generator unit includes an ozone generator fan;
b. a control unit, wherein the control unit is configured to control the ozone generator unit;
c. an occupancy sensor connected to control unit, wherein the occupancy sensor is configured to shut down the ozone generator when the occupancy sensor is activated;
d. an ozone exhaust fan, wherein the control unit controls the ozone exhaust fan;
an ozone destruct module containing ozone destruct catalyst, wherein the ozone destruct module is connected to the ozone exhaust fan;
a remote control, wherein the remote control is connected to a remote control connector that is electrically connected to the control unit, wherein the remote control further includes a power switch having a power switch warning light, an ozone generator light, and a safe entry light; and
a remote control cable harness connecting the remote control to the control unit.

2. The room sanitizer of claim 1, wherein the control unit further includes an ozone timer relay configured to set a time for operating the ozone generator.

3. The room sanitizer of claim 1, wherein the control unit further includes an ozone destruct timer relay configured to set a time for operating the ozone exhaust fan.
